(19) United States
(12) Patent Application Publication
(10) Pub. No.: US 2008/0227836 A1
(43) Pub. Date: Sep. 18, 2008

(54) Stable Solid Oral Dosage Forms of Valsartan

(75) Inventors: Makarand Krishnakumar Avachat, Maharashtra (IN); Nikhil Prabhakar Malewar, Maharashtra (IN); Umesh Nandkumar Khatavkar, Maharashtra (IN)

Correspondence Address: MERCHANT & GOULD PC, P.O. BOX 2903, MINNEAPOLIS, MN, 55402-0903, US

(73) Assignee: Lupin Ltd, Maharashtra (IN)

(21) Appl. No.: 12/092,009

(22) PCT Filed: Oct. 26, 2006
(86) PCT No.: PCT/IN2006/000421

§ 371 Date: Apr. 29, 2008

(30) Foreign Application Priority Data
Oct. 31, 2005 (IN) 985/KOL/2005

(51) Int. Cl. A61K 31/41; A61P 9/00

(52) U.S. Cl. 514/381

(57) ABSTRACT

A stable solid oral dosage form comprising valsartan or a pharmaceutically acceptable salt thereof and pharmaceutically acceptable additives such as sugar (derivatives) and cellulose (derivatives). The active agent is present in an amount less than 35% by weight based on the total weight of the solid oral dosage form. Disclosed is also a process of forming a valsartan-containing composition; which process comprises blending valsartan with pharmaceutically acceptable additives, dry compressing, milling and screening said mixture to obtain granules. Said granules are compressed into tablets and are subsequently coated.

### FIELD OF INVENTION

The present invention relates to stable solid oral dosage form comprising valsartan and a process of forming the same.

### BACKGROUND OF INVENTION

Valsartan is an angiotensin II antagonist and is known to be effective in the treatment of congestive heart failure and reducing blood pressure irrespective of age, sex or race and is also well tolerated. It has also been approved to treat people after heart attacks.

U.S. Pat. No. 5,399,578 describes the preparation of valsartan and its pharmaceutically acceptable salt.

U.S. Pat. Nos. 6,294,197, 6,485,745 and 6,858,228 describe a solid oral dosage form of valsartan and optionally hydrochlorthiazide (HCTZ) prepared by compression method having more than 35% by weight based on total weight of the compressed solid oral dosage form, of the active ingredient. Valsartan tablets of 80 mg, 40 mg, 160 mg and 320 mg strengths are available in the market.

U.S. patent application No: 2003/0152620 relates to solid oral dosage forms comprising more than about 65% by weight of valsartan or a pharmaceutically acceptable salt thereof or hydrate thereof. The compositions are at least 1.2 times more bioavailable than conventional valsartan capsule and having a Cmax of about at least 0.77 mg/l, e.g. up to 3.5 mg/l, e.g. 1.3 mg/l when administered as a dose of 40 mg in a single dose human bioavailability study.

We have developed novel solid oral dosage forms of valsartan, which are bioequivalent to the commercially available formulation of valsartan. Further, we have also found that this solid oral dosage form is stable at accelerated conditions of temperature and humidity.

### OBJECT OF THE INVENTION

According to the invention, there is provided a stable solid oral dosage form comprising

a) an active agent comprising an effective amount of valsartan and its pharmaceutically acceptable salt thereof; and
b) pharmaceutically acceptable additives.
wherein the active agent is present in an amount less than 35% by weight, based on the total weight of the stable solid oral dosage form.

Another object of the present invention is to provide a method of achieving bioequivalence between an immediate release coated tablets comprising valsartan or pharmaceutically acceptable salt thereof and the commercially available immediate release tablets, the said tablet being marketed under the brand name of ‘DIOVAN®’.

### DETAILED DESCRIPTION OF THE INVENTION

The present invention is related to a stable solid oral dosage form comprising
a) an active agent comprising an effective amount of valsartan or a pharmaceutically acceptable salt thereof; and
b) pharmaceutically acceptable additives suitable for the preparation of solid oral dosage forms,
   wherein the active agent is present in an amount less than 35% by weight based on the total weight of the solid oral dosage form.

“Solid oral dosage form” includes granules, pellets, tablets, capsules and the like prepared by conventional methods well known to a person skilled in the art.

By “effective amount”, it is meant that the amount of active agent, which halts or reduces the progress of the condition being treated or which otherwise completely or partly cures or acts palliatively on the condition. A person skilled in the art can easily determine such an amount by routine experimentation and with an undue burden.

In a stable solid dosage form according to the invention wherein, active agent consists entirely of valsartan or a pharmaceutically acceptable salt thereof, it is preferred if the active agent is present in the amount of from 10-320 mg e.g. 40, 80, 160 or 320 mg.

The solid oral dosage form may further comprise pharmaceutically acceptable excipients known in the art. These include but are not limited to disintegrants, binders, lubricants, glidants, fillers, diluents and the like.

The amounts of additive employed will depend upon how much active agent is to be used. One excipient can perform more than one function.

Disintegrants, which include but are not limited to, cross linked polyvinylpyrolidone (crospovidone, polyplasdoneXL, kollidon CL); starches such as maize starch and dried starch sodium starch glycolate; gums such as alginic acid, sodium alginate, guar gum; croscarmellose sodium; cellulose products such as microcrystalline cellulose and its salts, microfine cellulose, low substituted hydroxypropylcellulose and mixtures thereof. Most preferably disintegrants are crosslinked polyvinylpyrrolidone, crosslinked carboxymethylcellulose and crosslinked sodium carboxymethylcellulose.

Binders, which include, but are not limited to, alkylcelluloses such as methyl cellulose, ethyl cellulose; hydroxyalkylcelluloses such as hydroxypropylcellulose, low substituted hydroxypropylcellulose, hydroxypropyl methylcellulose, sodium carboxymethylcellulose; microcrystalline cellulose; starches, e.g. potato starch, wheat starch, corn starch, pregelatinised maize starch; or polyvinylpyrrolidone e.g. products known under the registered trade marks Avicel, Filtrak, Heweten or Pharmacel.

Lubricants may be selected from those conventionally known in the art such as Mg, Al or Ca stearate, polyethylene glycol and talc.

Glidants include colloidal silica, powdered cellulose, talc, tribasic calcium phosphate and the like.

Fillers or diluents, which include, but are not limited to confectioner's sugar, compressible sugar, dextrates, dextrin, dextrose, mannitol, sucrose, starch, lactose, dicalcium phosphate, xylitol, sorbitol, talc, micro-crystalline cellulose and the like can be used.

One or more of these additives can be selected and used by the skilled artisan having regard to the particular desired properties of the solid oral dosage form. The amount of each type of additive employed, e.g. glidant, binder, disintegrant, filler or diluent and lubricant may vary within ranges conventional in the art.

The solid dosage form of the invention, wherein the core can be formed by various methods known in the art such as by dry granulation, wet granulation, direct compression, extrusion spheronization, layering and the like.

In a preferred embodiment, there is provided a process of making the stable solid oral dosage forms as hereinabove described comprising the steps of
i) blending the active agent and pharmaceutically acceptable additives,
ii) subjecting the blend to slugging/compaction to form a coprimate
iii) converting the coprimate to form granules and
iv) compressing the granules to form the solid oral dosage form.

Compaction of the blend into coprimate may be carried out using a slugging technique or preferably, roller compaction. Roller compaction apparatus is conventional and essentially utilizes two rollers, which roll towards each other. Hydraulic ram forces one of the rollers against the other to exert a compacting force against the dry blend fed into the roller compactor via a screw conveyor system. The milling of the granules may be carried out according to conventional milling methods. The compression of the granulates to tablet cores can be carried out in a conventional tabletting machine, eccentric tabletting machine or a rotary compression machine.

The tablets were further coated by using any of the conventional coating techniques, such as pan or perforated pan, well known to the persons skilled in the art.

These coating layers comprise of one or more excipients selected from the group comprising coating agents, opacifiers, taste-masking agents, colouring agents, antitacking agents and the like.

Coating agents which are useful in the coating process, include, but are not limited to, polysaccharides such as maltodextrin, alkyl celluloses such as methyl or ethyl cellulose, hydroxyalkylcelluloses (e.g. hydroxypropylcellulose or hydroxypropylmethylcelluloses); polyvinylpyrrolidone, polyvinyl alcohol, copolymers of vinylpyrrolidone and vinyl acetate (e.g. marketed under the brand name of Plasdone) and polymers based on methacrylic acid such as those marketed under the brand name of Eudragit. These may be applied from aqueous or non-aqueous systems or combinations of aqueous and non aqueous systems as appropriate. Additives can be included along with the film formers to obtain satisfactory films. These additives can include plasticizers such as dibutyl phthalate, triethyl citrate, polyethylene glycol and the like, antitacking agents such as talc, stearic acid, magnesium stearate and colloidal silicon dioxide and the like, surfactants such as polysorbates and sodium lauryl sulphate and opacifying agents such as titanium dioxide and the like. All these excipients can be used at levels well known to the persons skilled in the art.

The following examples serve to illustrate the invention.

### EXAMPLES

| | % w/w | % w/w | % w/w | % w/w |
|---|---|---|---|---|
| | Example No: | | | |
| Ingredients | 1 | 2 | 3 | 4 |
| Valsartan | 32.00 | 30.77 | 30.77 | 32.00 |
| Microcrystalline Cellulose | 60.15 | 61.38 | 61.38 | 60.15 |
| Crosslinked polyvinylpyrrolidone | 4.50 | 4.50 | 4.50 | 4.50 |
| Colloidal Silicon Dioxide | 2.10 | 2.10 | 2.10 | 2.10 |
| Magnesium Stearate | 1.25 | 1.25 | 1.25 | 1.25 |
| Opadry | QS | QS | QS | QS |

### Brief Manufacturing Process

1. The sifted components except a part of microcrystalline cellulose, crosslinked polyvinylpyrollidone and colloidal silicon dioxide are blended in a suitable blender.
2. The blended material is compacted to form slugs/compacts.
3. The compacted material is milled and sieved again to form granules. The remaining portion of the microcrystalline cellulose, crosslinked polyvinylpyrollidone and colloidal silicon dioxide are added and blended in a suitable blender.
4. The prepared granules are lubricated and compressed into the tablets.
5. The tablets are then coated using aqueous Opadry.

All other strengths 40 mg, 80 mg, and 160 mg are exactly step down process. The slugging or roller compaction method was used.

### Stability Study

The formulation prepared according to the example 1 is subjected to stability studies at accelerated conditions of temperature and humidity of 40° C. and 75% RH. Results of these stability studies are summarized in the table 1.

TABLE 1
Stability Data

| | Package Type | | | | | |
|---|---|---|---|---|---|---|
| | | | | PVC/PE/PVDC-Aluminium | | |
| Related | HDPE | | | Blister | | |
| Substances | Initial | 1 M | 2 M | Initial | 1 M | 2 M |
| N-Isobutyl | ND | BDL | BDL | ND | BDL | BDL |
| Valsartan | | | | | | |
| Impurity B | ND | 0.070 | 0.073 | ND | 0.063 | 0.076 |
| Impurity C | ND | BDL | BDL | ND | BDL | BDL |
| Assay | 99.14 | 97.22 | 97.70 | 98.63 | 98.87 | 97.12 |

BDL = Below Detectable Limit
ND = Not detectable
HDPE = High density Polyethylene
PVC = Polyvinyl Chloride
PE = Polyethylene
PVDC = Polyvinyl Dichloride
M = Month

Dissolution Study

The in vitro specifications for generic products should be established based on a dissolution profile. In the case of a generic drug product, the dissolution specifications are generally the same as the reference listed drug.

A dissolution test was carried out in four different medias: 0.1NHCl, purified water, acetate buffer pH4.5 and 0.067 M phosphate buffer pH6.8. The following compositions were tested: immediate release tablets comprising of 320 mg of valsartan, prepared according to example 1 as test and DIOVAN® having Valsartan 320 mg, by Novartis as reference.

To determine the similarity between the dissolution profiles of the test and reference product a simple model independent approach, that is f₂ (similarity factor), was carried out. As per US FDA, f₂ values should lie between 50-100 for rendering two dissolution profiles similar. The results obtained are summarized below in table 2.

TABLE 2
The f₂ values of dissolution profiles of the Valsartan immediate
release tablets against commercially available immediate release tablets
DIOVAN ® in four different medias.

| Media | f₂ value |
|---|---|
| 0.1N HCl | 92.87 |
| Purified water | 91.91 |
| Acetate buffer pH4.5 | 50.92 |
| 0.067 M Phosphate buffer pH6.8 | 56.27 |

The above results clearly show that the f₂ values for all the four medias mentioned above are within the limits of 50-100 as established by the US FDA for claiming similarity between the dissolution profiles of the test and reference product.

### Bioequivalence Study

A bioequivalence study was carried out in 10 healthy human volunteers receiving single dose of valsartan in fed and fasted state using immediate release tablets comprising of 320 mg of valsartan, prepared according to example 1, as test and DIOVAN® having Valsartan 320 mg, by Novartis, as reference. Study was monitored in terms of the pharmacokinetic parameters Cₘₐₓ and AUC. AUCs are plots of plasma concentrations of valsartan along the ordinate (Y-axis) against time on the abscissa (X-axis). Generally, the values for AUC represent a number of values taken from all the subjects in a population and are, therefore, mean values averaged over the entire population. Cₘₐₓ, the observed maximum in a plot of plasma level concentration of valsartan (Y-axis) versus time (X-axis) is likewise an average value.

The ratios of the log transformed mean values for Cₘₐₓ and AUC for the test and reference product (T/R ratio) is a measure of the bioequivalence between the test and reference product. Values between 80 and 125% for the 90% confidence intervals of these ratios indicate bioequivalence as recommended by the US FDA.

Bioequivalence data for the Valsartan tablets against the commercially available tablets “DIOVAN” is shown below in Table 3 and 4.

TABLE 3
Fed BE study data of Valsartan immediate release tablets against
commercially available immediate release tablets DIOVAN ®;
n = 10

| | | Log |
|---|---|---|
| | | Transformed |
| | | T/R (%) of |
| PK | Mean Plasma | Geometric Least |
| Parameters | Concentration | Square Mean |
| Cₘₐₓ | 6139 ng/ml ± 1789 | 114.76 |
| AUC₍₀₋ₜ₎ | 4513 ng * hr/ml ± 15416 | 115.42 |
| AUC_{(0-∞)} | 46093 ng * hr/ml ± 16400 | 114.98 |

TABLE 4
Fasted BE study data of Valsartan immediate release tablets
against commercially available immediate release tablets
“DIOVAN ®” n = 10

| | | Log |
|---|---|---|
| | | Transformed |
| | | T/R (%) of |
| | | Geometric |
| PK | Mean Plasma | Least Square |
| Parameters | Concentration | Mean |
| Cₘₐₓ | 5978 ng/ml ± 3108 | 102.58 |
| AUC₍₀₋ₜ₎ | 35848 ng * hr/ml ± 13953 | 100.08 |
| AUC_{(0-∞)} | 36893 ng * hr/ml ± 13975 | 100.06 |

Cₘₐₓ = Maximum plasma concentration
AUC₍₀₋ₜ₎ = Area under the plasma concentration time curve from time 0 to t
AUC_{(0-∞)} = Area under the plasma concentration time curve from time 0 to ∞

As can be seen from the data above in Tables 3 and 4, a Log transformed T/R (%) ratio of Geometric Least Square mean in fed and fasted in case of Cₘₐₓ, AUC₍₀₋ₜ₎ and AUC_{(0-∞)} was well within the limits of 80-125% as established by the US FDA for claiming bioequivalence between a test and reference product.

1. A stable solid oral dosage form comprising
a) an active agent comprising an effective amount of valsartan or a pharmaceutically acceptable salt thereof; and
b) pharmaceutically acceptable additives suitable for the preparation of solid oral dosage forms,
wherein the active agent is present in an amount less than 35% by weight based on the total weight of the solid oral dosage form.

2. A stable solid oral dosage form according to claim 1, wherein valsartan is present in a unit dose from 40 mg to 320 mg.

3. A stable solid oral dosage form according to claim 1, pharmaceutically acceptable additives selected from the group comprising fillers or diluents, binders, lubricants, glidants and disintegrants.

4. The stable solid oral dosage form of claim 1, wherein the diluent is one or more selected from the group comprising confectioner's sugar, compressible sugar, dextrates, dextrin, dextrose, mannitol, sucrose, starch, lactose, dicalcium phosphate, xylitol, sorbitol, talc, micro-crystalline cellulose or mixtures thereof.

5. The stable solid oral dosage form of claim 1, wherein the binder is one or more selected from the group comprising methyl cellulose, hydroxypropylcellulose, low substituted hydroxypropylcellulose, hydroxypropyl methylcellulose, sodium carboxymethylcellulose, microcrystalline cellulose, potato starch, wheat starch, corn starch, pregelatinised maize starch, polyvinylpyrrolidone or mixtures thereof.

6. The stable solid oral dosage form of claim 1, wherein the lubricant is one or more selected from the group comprising Mg, Al or Ca stearate, polyethyleneglycol, talc or mixtures thereof.

7. The stable solid oral dosage form of claim 1, wherein the glidant is one or more selected from the group comprising colloidal silica, powdered cellulose, talc, tribasic calcium phosphate or mixtures thereof.

8. The stable solid oral dosage form of claim 1, wherein the disintegrant is one or more selected from the group comprising cross linked polyvinylpyrrolidone, maize starch, dried starch, sodium starch glycolate, alginic acid, sodium alginate, guar gum, croscarmellose sodium, microcrystalline cellulose and its salts, microfine cellulose, low substituted hydroxypropylcellulose and mixtures thereof.

9. A stable solid oral dosage form as in claim 1, is further coated with coating layer comprising coating agents, plasticizers, antitacking agents, surfactants, coloring agents, opacifiers or mixtures thereof.

10. A process of preparation of stable solid oral dosage form of valsartan, the said process comprising the steps of blending valsartan with the other excipients, dry compression, milling and screening to obtain granules, said granules being subsequently compressed into tablets and coated.

11. A stable solid oral dosage form as in claim 1 is in the form of tablets.

12. A method of achieving bioequivalence between an immediate release valsartan coated tablets prepared as per example 1, having 320 mg of valsartan and a commercially available 320 mg immediate release tablets of valsartan, the said tablet being marketed under the brand name of ‘DIOVAN®’, the method comprising formulating the composition in the form of immediate release tablets wherein valsartan is present in an amount less than 35% by weight based on the total weight of the solid oral dosage form.
